SEQUENCE LISTING (3 sequences)

SEQ ID NO: 1 - protein, 416 aa, Homo sapiens
MATEPPSPLRVEAPGPPEMRTSPAIESTPEGTPQPAGGRLRFLNGCVPLSHQVAGHMYGKDKVGILQHPDGTVLKQLQPPPRGPRELEFYNMVYAADCFDGVLLELRKYLPKYYGIWSPPTAPNDLYLKLEDVTHKFNKPCIMDVKIGQKSYDPFASSEKIQQQVSKYPLMEEIGFLVLGMRVYHVHSDSYETENQHYGRSLTKETIKDGVSRFFHNGYCLRKDAVAASIQKIEKILQWFENQKQLNFYASSLLFVYEGSSQPTTTKLNDRTLAEKFLSKGQLSDTEVLEYNNNFHVLSSTANGKIESSVGKSLSKMYARHRKIYTKKHHSQTSLKVENLEQDNGWKSMSQEHLNGNVLSQLEKVFYHLPTGCQEIAEVEVRMIDFAHVFPSNTIDEGYVYGLKHLISVLRSILDN

SEQ ID NO: 2 - protein, 407 aa, Homo sapiens
MEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

SEQ ID NO: 3 - protein, 420 aa, Homo sapiens
MLHFHYYDTSNKIMEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

(19) United States
(12) Patent Application Publication
(10) Pub. No.: US 2024/0238384 A1
(43) Pub. Date: Jul. 18, 2024

(54) Treatment Of B27-Negative Uveitis With Inositol Polyphosphate Multikinase (IPMK) Therapeutic Agents Or Indoleamine 2,3-dioxygenase 2 (IDO2) Agonists

(71) Applicant: Regeneron Pharmaceuticals, Inc., Tarrytown, NY (US)

(72) Inventors: Sahar Gelfman, Tarrytown, NY (US); Arden Moscati, Tarrytown, NY (US); Aris Baras, Tarrytown, NY (US); Eli A. Stahl, Tarrytown, NY (US); Giovanni Coppola, Tarrytown, NY (US); Ann Ligocki, Tarrytown, NY (US); Carmelo Romano, Tarrytown, NY (US); Santiago Mendez Huergo, Tarrytown, NY (US); Jonathan Weyne, Tarrytown, NY (US); Tave Van Zyl, Tarrytown, NY (US); Rujin Wang, Tarrytown, NY (US); Charles Miller, Tarrytown, NY (US)

(21) Appl. No.: 18/413,204

(22) Filed: Jan. 16, 2024

Related U.S. Application Data
(60) Provisional application No. 63/439,520, filed on Jan. 17, 2023.

(51) Int. Cl. A61K 38/45; A61K 38/44; C12Q 1/6883

(52) U.S. Cl. CPC A61K 38/45; A61K 38/44; C12Q 1/6883; C12Y 113/11052; C12Y 207/01151; C12Q 2600/118

(57) ABSTRACT

Methods of treating B27-negative subjects having uveitis with an Inositol Polyphosphate Multikinase (IPMK) therapeutic agent and/or an Indoleamine 2,3-dioxygenase 2 (IDO2) agonist, and methods of identifying subjects having an increased risk of developing uveitis are described herein.

### REFERENCE TO SEQUENCE LISTING

This application includes a Sequence Listing filed electronically as an XML file named 381204101SEQ, created on Jan. 12, 2024 with a size of 4,756 bytes. The Sequence Listing is incorporated herein by reference.

### FIELD

The present disclosure relates generally to the treatment of B27-negative subjects having uveitis, such as anterior uveitis, with an Inositol Polyphosphate Multikinase (IPMK) therapeutic agent and/or an Indoleamine 2,3-dioxygenase 2 (IDO2) agonist, and methods of identifying subjects having an increased risk of developing uveitis.

### BACKGROUND

Anterior uveitis (AU) is an inflammation of the middle layer of the eye. This middle layer includes the iris (colored part of the eye) and adjacent tissue, known as the ciliary body. This intraocular inflammatory disease can be categorized by etiology, in infectious or non-infectious, and by anatomy, in anterior, intermediate, posterior or panuveitis. Non-infectious uveitis represents the majority of the cases in the developed world, with a prevalence of 121 per 100,000 in the United States (Joltikov et al., Epidemiology and Risk Factors in Non-infectious Uveitis: A Systematic Review, Front Med (Lausanne) 2021, 8, 695904. Among them, AU, characterized by inflammation of the iris and/or ciliary body, is the most common type of non-infectious uveitis, representing 47.5 to 93% of cases. AU predominately affects individuals at younger ages than many eye diseases, with a mean age of onset less than 40 years of age (de Smet et al., Prog. Retin. Eye Res., 2011, 30, 452-470; and Rothova et al., Br. J. Ophthalmol., 1996, 80, 332-336). It is classified as a rare disease, however, with an estimated prevalence of approximately 38 cases per 100,000 people (Chang et al., Ocul, Immunol, Inflamm., 2002, 10, 263-279; and Henderly et al., Am. J. Ophthalmol., 1987, 103, 131-136).

AU can result from a trauma to the eye, such as being hit in the eye or having a foreign body in the eye. It can also be associated with general health problems such as rheumatoid arthritis, syphilis, tuberculosis, sarcoid, viral (herpes simplex, herpes zoster, cytomegalovirus) or idiopathic, which is no obvious underlying cause. Acute anterior uveitis (AAU) involves inflammation of the iris and ciliary body of the eye. AAU occurs both in isolation and as an extra-articular feature of ankylosing spondylitis (AS). It is potentially sight threatening and can cause ophthalmological sequelae such as cataracts, posterior synechiae, and glaucoma. Uveitis accounts for about 10% of those individuals with severe vision impairment and blindness. Eye redness, acute pain and blurred vision associated with cellular infiltration in the anterior chamber are among the main symptoms of AU. AU is usually associated as a complication of spondyloarthropathies (SpAs), like ankylosing spondylitis (AS), psoriatic arthritis (PsA) and inflammatory bowel disease (IBD).

Inositol Polyphosphate Multikinase (IPMK) has catalytic activity that yields water-soluble inositol polyphosphates that regulate many cellular functions, including cell growth, apoptosis, cell migration, endocytosis, and cell differentiation. IPMK is considered a signaling hub in mammalian cells that coordinates the activity of various signaling networks including regulating the TLR-induced innate immunity (Lee et al., Mol. Cells, 2021, 44, 187-194).

Indoleamine 2,3-dioxygenase 2 (IDO2) is a LoF tolerant gene that exhibits a pLI score of 0 and O/E=0.81 (0.54-1.25) (Karczewski et al., Nature, 2020, 581, 434-443). IDO2 is a known immune-modulator and has been shown to was play a role for the differentiation of regulatory T cells in vitro and play a pro-inflammatory role in the development of B cell-mediated autoimmune arthritis (Merlo et al., Clin. Pathol. 2020, 13, 2632010X20951812; and Merlo et al., Clin. Immunol., 2017, 179, 8-16). It may be likely that the loss of IDO2 disrupts the T-cell regulation and affects the T-Cell mediated response in the anterior chamber, eventually leading to AU.

### SUMMARY

The present disclosure provides methods of treating uveitis in a subject that is HLA-B27-negative, the methods comprising administering an IPMK therapeutic agent or an IDO2 agonist.

The present disclosure also provides methods of treating iridocyclitis in a subject that is HLA-B27-negative, the methods comprising administering an IPMK therapeutic agent or an IDO2 agonist.

The present disclosure also provides methods of treating iritis in a subject that is HLA-B27-negative, the methods comprising administering an IPMK therapeutic agent or an IDO2 agonist.

The present disclosure also provides methods of treating a subject with a uveitis therapeutic agent, wherein the subject has B27-negative uveitis, the methods comprising: determining whether the subject has an IPMK variant nucleic acid molecule or an IDO2 variant nucleic acid molecule by: obtaining or having obtained a biological sample from the subject; and performing or having performed a sequence analysis on the biological sample to determine if the subject has a genotype comprising the IPMK variant nucleic acid molecule and/or the IDO2 variant nucleic acid molecule; and administering or continuing to administer the uveitis therapeutic agent in a standard dosage amount to a subject that is IPMK reference and IDO2 reference; administering or continuing to administer the uveitis therapeutic agent in an amount that is the same as or less than a standard dosage amount to a subject that is heterozygous or homozygous for the IPMK variant nucleic acid molecule, and/or administering an IPMK therapeutic agent to the subject; and administering or continuing to administer the uveitis therapeutic agent in an amount that is the same as or less than a standard dosage amount to a subject that is heterozygous or homozygous for the IDO2 variant nucleic acid molecule, and/or administering an IDO2 agonist to the subject; wherein the presence of a genotype having the IPMK variant nucleic acid molecule and/or the IDO2 variant nucleic acid molecule indicates the subject has an increased risk of developing uveitis.

The present disclosure also provides methods of identifying a B27-negative subject having an increased risk of developing uveitis, the methods comprising: determining or having determined the presence or absence of an IPMK variant nucleic acid molecule or an IDO2 variant nucleic acid molecule in a biological sample obtained from the subject; wherein: when the subject is IPMK reference and IDO2 reference, then the subject does not have an increased risk of developing uveitis; and when the subject is heterozygous or homozygous for an IPMK variant nucleic acid molecule or an IDO2 variant nucleic acid molecule, then the subject has an increased risk of developing uveitis.

The present disclosure also provides uveitis therapeutic agents for use in the treatment of uveitis in a subject that is B27-negative, and the subject is identified as having an IPMK variant nucleic acid molecule and/or an IDO2 variant nucleic acid molecule.

The present disclosure also provides IPMK therapeutic agents for use in the treatment of uveitis in a B27-negative subject that comprises an IPMK variant nucleic acid molecule.

The present disclosure also provides IDO2 agonists for use in the treatment of uveitis in a B27-negative subject that comprises an IDO2 variant nucleic acid molecule.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is not intended that any method or aspect set forth herein be construed as requiring that its steps be performed in a specific order. Accordingly, where a method claim does not specifically state in the claims or descriptions that the steps are to be limited to a specific order, it is not intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or other tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

Variants in the IPMK gene that result in reduced expression of IPMK or that result in an IPMK predicted loss-of-function polypeptide associated with an increased risk of developing uveitis, such as anterior uveitis, acute anterior uveitis, iridocyclitis, iritis, and pan-uveitis, in humans who are B27-negative has been identified in accordance with the present disclosure. In addition, variants in the IDO2 gene that result in reduced expression of IDO2 or that result in an IDO2 predicted loss-of-function polypeptide associated with an increased risk of developing uveitis, such as anterior uveitis, acute anterior uveitis, iridocyclitis, iritis, and pan-uveitis, in humans has been identified in accordance with the present disclosure. Altogether, the genetic analyses described herein surprisingly indicate that the IPMK gene and/or the IDO2 gene and, in particular, variants in these genes, associate with an increased risk of developing uveitis. Therefore, subjects that are heterozygous or homozygous for an IPMK variant nucleic acid molecule and/or an IDO2 variant nucleic acid molecule that have an increased risk of developing uveitis, such as anterior uveitis, acute anterior uveitis, iridocyclitis, iritis, and pan-uveitis, may be treated such that the uveitis is prevented, the symptoms thereof are reduced, and/or development of symptoms is repressed. Accordingly, the present disclosure provides methods of leveraging the identification of such variants in subjects to identify or stratify risk in such subjects of developing uveitis, such as anterior uveitis, acute anterior uveitis, iridocyclitis, iritis, and pan-uveitis, or to diagnose subjects as having an increased risk of developing uveitis, such as anterior uveitis, acute anterior uveitis, iridocyclitis, iritis, and pan-uveitis, such that subjects at risk or subjects with active disease may be treated accordingly.

For purposes of the present disclosure, any particular subject can be categorized as having one of three IPMK genotypes: i) IPMK reference; ii) heterozygous for an IPMK variant nucleic acid molecule; or iii) homozygous for an IPMK variant nucleic acid molecule. A subject is IPMK reference when the subject does not have a copy of an IPMK variant nucleic acid molecule. A subject is heterozygous for an IPMK variant nucleic acid molecule when the subject has a single copy of an IPMK variant nucleic acid molecule. As used herein, an IPMK variant nucleic acid molecule is any IPMK nucleic acid molecule (such as, a genomic nucleic acid molecule, an mRNA molecule, or a cDNA molecule) encoding an IPMK polypeptide having a partial loss-of-function, a complete loss-of-function, a predicted partial loss-of-function, or a predicted complete loss-of-function, or any IPMK nucleic acid molecule that results in decreased expression of IPMK polypeptide. A subject who has an IPMK variant nucleic acid molecule is hypomorphic for IPMK. In any of the embodiments described herein, the IPMK variant genomic nucleic acid molecule may include one or more variations at any of the positions of chromosome 10 (i.e., positions 58,191,517-58,267,894) using the nucleotide sequence of the IPMK reference genomic nucleic acid molecule in the GRCh38/hg38 human genome assembly (see, ENSG00000151151.6, ENST00000373935.4 annotated in the in the Ensembl database (URL: world wide web at “useast.ensembl.org/Homo_sapiens/Gene/Summary?g=ENSG00000151151;r=10:58191517-58267894; t=ENST00000373935”) as a reference sequence. For example, the IPMK variant nucleic acid molecule encoding an IPMK predicted loss-of-function polypeptide can be any IPMK genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule. A subject is homozygous for an IPMK variant nucleic acid molecule when the subject has two copies of an IPMK variant nucleic acid molecule.

For purposes of the present disclosure, any particular subject can be categorized as having one of three IDO2 genotypes: i) IDO2 reference; ii) heterozygous for an IDO2 variant nucleic acid molecule; or iii) homozygous for an IDO2 variant nucleic acid molecule. A subject is IDO2 reference when the subject does not have a copy of an IDO2 variant nucleic acid molecule. A subject is heterozygous for an IDO2 variant nucleic acid molecule when the subject has a single copy of an IDO2 variant nucleic acid molecule. As used herein, an IDO2 variant nucleic acid molecule is any IDO2 nucleic acid molecule (such as, a genomic nucleic acid molecule, an mRNA molecule, or a cDNA molecule) encoding an IDO2 polypeptide having a partial loss-of-function, a complete loss-of-function, a predicted partial loss-of-function, or a predicted complete loss-of-function, or any IDO2 nucleic acid molecule that results in decreased expression of IDO2 polypeptide. A subject who has an IDO2 variant nucleic acid molecule is hypomorphic for IDO2. In any of the embodiments described herein, the IDO2 variant genomic nucleic acid molecule may include one or more variations at any of the positions of chromosome 8 (i.e., positions 39,934,651-40,016,392) using the nucleotide sequence of the IDO2 reference genomic nucleic acid molecule in the GRCh38/hg38 human genome assembly (see, ENSG00000188676.15, ENST00000502986.4 annotated in the in the Ensembl database (URL: world wide web at “useast.ensembl.org/Homo_sapiens/Gene/Summary?g=ENSG00000188676;r=8:39934614-40016392; transcript=ENST00000502986.4”) as a reference sequence. For example, the IDO2 variant nucleic acid molecule encoding an IDO2 predicted loss-of-function polypeptide can be any IDO2 genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule. A subject is homozygous for an IDO2 variant nucleic acid molecule when the subject has two copies of an IDO2 variant nucleic acid molecule.

For subjects that are genotyped or determined to be heterozygous or homozygous for an IPMK variant nucleic acid molecule and/or an IDO2 variant nucleic acid molecule, such subjects have an increased risk of developing uveitis, such as anterior uveitis, acute anterior uveitis, iridocyclitis, iritis, and pan-uveitis. For subjects that are genotyped or determined to be heterozygous or homozygous for an IPMK variant nucleic acid molecule, such subjects can be treated with an IPMK therapeutic agent. For subjects that are genotyped or determined to be heterozygous or homozygous for an IDO2 variant nucleic acid molecule, such subjects can be treated with an IDO2 agonist.

For purposes of the present disclosure, the treated subjects described herein are B27-negative (negative for HLA-B27). Such subjects have no copies of the HLA-B27 allele in their genome.

In any of the embodiments described throughout the present disclosure, the IPMK variant nucleic acid molecule can be any IPMK nucleic acid molecule (such as, for example, genomic nucleic acid molecule, mRNA molecule, or cDNA molecule) encoding an IPMK polypeptide having a partial loss-of-function, a complete loss-of-function, a predicted partial loss-of-function, or a predicted complete loss-of-function, or any IPMK nucleic acid molecule that results in decreased expression of IPMK polypeptide. In any of the embodiments described herein, the IPMK variant nucleic acid molecule can be any nucleic acid molecule (such as, a genomic nucleic acid molecule, an mRNA molecule, or a cDNA molecule) that is a missense variant, a splice-site variant, a stop-gain variant, a start-loss variant, a stop-loss variant, a frameshift variant, an in-frame indel variant, or a variant that encodes a truncated IPMK polypeptide. For example, the IPMK variant nucleic acid molecule can be any IPMK genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule.

In any of the embodiments described throughout the present disclosure, the IDO2 variant nucleic acid molecule can be any IDO2 nucleic acid molecule (such as, for example, genomic nucleic acid molecule, mRNA molecule, or cDNA molecule) encoding an IDO2 polypeptide having a partial loss-of-function, a complete loss-of-function, a predicted partial loss-of-function, or a predicted complete loss-of-function, or any IDO2 nucleic acid molecule that results in decreased expression of IDO2 polypeptide. In any of the embodiments described herein, the IDO2 variant nucleic acid molecule can be any nucleic acid molecule (such as, a genomic nucleic acid molecule, an mRNA molecule, or a cDNA molecule) that is a missense variant, a splice-site variant, a stop-gain variant, a start-loss variant, a stop-loss variant, a frameshift variant, an in-frame indel variant, or a variant that encodes a truncated IDO2 polypeptide. For example, the IDO2 variant nucleic acid molecule can be any IDO2 genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule.

In any of the embodiments described throughout the present disclosure, the uveitis is anterior uveitis, acute anterior uveitis, iridocyclitis, iritis, or pan-uveitis. In any of the embodiments described throughout the present disclosure, the uveitis is anterior uveitis. In any of the embodiments described throughout the present disclosure, the uveitis is acute anterior uveitis. In any of the embodiments described throughout the present disclosure, the anterior uveitis includes iridocyclitis. In any of the embodiments described throughout the present disclosure, the anterior uveitis includes iritis. In any of the embodiments described throughout the present disclosure, the uveitis is pan-uveitis. In any of the embodiments described throughout the present disclosure, the subject can have ankylosing spondylitis. In any of the embodiments described throughout the present disclosure, the subject does not have ankylosing spondylitis.

Symptoms of an anterior uveitis include, but are not limited to, severe redness in the eye, pain, dark floating spots in one's vision (so-called called “floaters”), light sensitivity, and or blurred vision.

The present disclosure provides methods of treating uveitis in a subject that is HLA-B27-negative, the method comprising administering an IPMK therapeutic agent or an IDO2 agonist. In some embodiments, the uveitis is anterior uveitis. In some embodiments, the uveitis is acute anterior uveitis. In some embodiments, the uveitis is pan-uveitis.

The present disclosure also provides methods of treating iridocyclitis in a subject that is HLA-B27-negative, the methods comprising administering an IPMK therapeutic agent or an IDO2 agonist.

The present disclosure also provides methods of treating iritis in a subject that is HLA-B27-negative, the methods comprising administering an IPMK therapeutic agent or an IDO2 agonist.

In some embodiments, the IPMK therapeutic agent comprises an agonist. In some embodiments, the IPMK therapeutic agent comprises an antagonist. In some embodiments, the IPMK therapeutic agent comprises an IPMK protein. In some embodiments, the IPMK protein comprises the amino acid sequence MATEPPSPLRVEAPGPPEMRTSPAIESTPEGTPQPA GGRLRFLNGCVPLSHQVAGHMYGKDKVGILQHPDGTVLKQLQPPPRGPRELEFYNMVYAADCFDGVLL ELRKYLPKYYGIWSPPTAPNDLYLKLEDVTHKFNKPCIMDVKIGQKSYDPFASSEKIQQQVSKYPLMEEIG FLVLGMRVYHVHSDSYETENQHYGRSLTKETIKDGVSRFFHNGYCLRKDAVAASIQKIEKILQWFENQKQ LNFYASSLLFVYEGSSQPTTTKLNDRTLAEKFLSKGQLSDTEVLEYNNNFHVLSSTANGKIESSVGKSLSKM YARHRKIYTKKHHSQTSLKVENLEQDNGWKSMSQEHLNGNVLSQLEKVFYHLPTGCQEIAEVEVRMIDF AHVFPSNTIDEGYVYGLKHLISVLRSILDN (SEQ ID NO:1) (NCBI Reference Sequence: NM_152230.5). In some embodiments, the IPMK therapeutic agent is delivered to a particular cellular population. In some embodiments, the cellular population is an antigen presenting cell, such as a macrophage. In some embodiments, the IPMK therapeutic agent comprises a flavonoid, such as, for example myricetin, quercetin, luteolin, kaempferol, isorhamnetin, diosmetin, rhamnetin, and apigenin. In some embodiments, the IPMK therapeutic agent comprises vilazodone. In some embodiments, the IPMK therapeutic agent comprises aurintricarboxylic acid. In some embodiments, the IPMK therapeutic agent comprises chlorogenic acid.

In some embodiments, the IDO2 agonist comprises an IDO2 protein. In some embodiments, the IDO2 protein comprises the amino acid sequence MEPHRPNVKTAVPLSLE SYHISEEYGFLLPDSLKELPDHYRPWMEIANKLPQLIDAHQLQAHVDKMPLLSCQFLKGHREQRLAHLVL SFLTMGYVWQEGEAQPAEVLPRNLALPFVEVSRNLGLPPILVHSDLVLTNWTKKDPDGFLEIGNLETIISF PGGESLHGFILVTALVEKEAVPGIKALVQATNAILQPNQEALLQALQRLRLSIQDITKTLGQMHDYVDPDI FYAGIRIFLSGWKDNPAMPAGLMYEGVSQEPLKYSGGSAAQSTVLHAFDEFLGIRHSKESGDFLYRMRD YMPPSHKAFIEDIHSAPSLRDYILSSGQDHLLTAYNQCVQALAELRSYHITMVTKYLITAAAKAKHGKPNH LPGPPQALKDRGTGGTAVMSFLKSVRDKTLESILHPRG (SEQ ID NO:2) (NCBI Reference Sequence: NM_194294.5 and NP_001382135.1) or MLHFHYYDTSNKIMEPHRPNVKTAVPLSLESYHISEEYGF LLPDSLKELPDHYRPWMEIANKLPQLIDAHQLQAHVDKMPLLSCQFLKGHREQRLAHLVLSFLTMGYV WQEGEAQPAEVLPRNLALPFVEVSRNLGLPPILVHSDLVLTNWTKKDPDGFLEIGNLETIISFPGGESLHG FILVTALVEKEAVPGIKALVQATNAILQPNQEALLQALQRLRLSIQDITKTLGQMHDYVDPDIFYAGIRIFLS GWKDNPAMPAGLMYEGVSQEPLKYSGGSAAQSTVLHAFDEFLGIRHSKESGDFLYRMRDYMPPSHKA FIEDIHSAPSLRDYILSSGQDHLLTAYNQCVQALAELRSYHITMVTKYLITAAAKAKHGKPNHLPGPPQALK DRGTGGTAVMSFLKSVRDKTLESILHPRG (SEQ ID NO:3) (Q6ZQW0-1 from Uniprot).

In some embodiments, the methods further comprise administering a uveitis therapeutic agent to the subject. Examples of uveitis therapeutic agents include, but are not limited to: ophthalmic steroids, such as prednisolone, prednisone, difluprednate, triamcinolone acetonide, fluoromethol, fluocinolone, or dexamethasone; immunosuppressants, such as azathioprine and cyclophosphamide; glucocorticoids, such as cortisone; and antirheumatics, such as adalimumab. Additional uveitis therapeutic agents include, but are not limited to, cyclopentolate, atropine, homatropine, corticotropin, gentamicin, corticotropin, loteprednol, tobramycin, atropine, sulfasalazine, hydrocortisone, neomycin, polymyxin b, bacitracin, and sulfacetamide sodium.

The present disclosure also provides compositions comprising any one or more of the IDO2 agonists and/or IPMK therapeutic agents described herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the methods of treatment further comprise detecting the presence or absence of an IPMK variant nucleic acid molecule and/or an IDO2 variant nucleic acid molecule in a biological sample obtained from the subject. Detecting the presence or absence of an IPMK variant nucleic acid molecule and/or an IDO2 variant nucleic acid molecule in a biological sample obtained from a subject and/or determining whether a subject has an IPMK variant nucleic acid molecule and/or an IDO2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the IPMK variant nucleic acid molecule and/or the IDO2 variant nucleic acid molecule can be present within a cell obtained from the subject. In some embodiments, the detecting step is carried out in vitro. In some embodiments, the detecting step comprises sequencing the entire nucleic acid molecule.

In some embodiments, the methods comprise administering a uveitis therapeutic agent in a standard dosage amount to a subject wherein the IPMK variant nucleic acid molecule is absent from the biological sample. In some embodiments, the methods comprise administering a uveitis therapeutic agent in a dosage amount that is the same as or less than a standard dosage amount to a subject that is heterozygous or homozygous for the IPMK variant nucleic acid molecule. In some embodiments, the IPMK variant nucleic acid molecule comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A (ENSG00000151151).

In some embodiments, the methods comprise administering a uveitis therapeutic agent in a standard dosage amount to a subject wherein the IDO2 variant nucleic acid molecule is absent from the biological sample. In some embodiments, the methods comprise administering a uveitis therapeutic agent in a dosage amount that is the same as or less than a standard dosage amount to a subject that is heterozygous or homozygous for the IDO2 variant nucleic acid molecule. In some embodiments, the IDO2 variant nucleic acid molecule comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C (ENSG00000188676).

In some embodiments, the treatment methods further comprise detecting the presence or absence of an IPMK predicted loss-of-function polypeptide and/or an IDO2 predicted loss-of-function polypeptide in a biological sample obtained from the subject. In some embodiments, when the subject does not have an IPMK predicted loss-of-function polypeptide and/or an IDO2 predicted loss-of-function polypeptide, the subject is administered a uveitis therapeutic agent in a standard dosage amount. In some embodiments, when the subject has an IPMK predicted loss-of-function polypeptide and/or an IDO2 predicted loss-of-function polypeptide, the subject is administered a uveitis therapeutic agent in a dosage amount that is the same as or less than a standard dosage amount, and/or an IPMK therapeutic agent and/or an IDO2 agonist.

The present disclosure also provides methods of treating a subject with a uveitis therapeutic agent. Such subjects are HLA-B27-negative. In some embodiments, the subject is at risk of developing uveitis, such as anterior uveitis. The methods comprise determining whether the subject has an IPMK variant nucleic acid molecule or an IDO2 variant nucleic acid molecule by: obtaining or having obtained a biological sample from the subject; and performing or having performed a sequence analysis on the biological sample to determine if the subject has a genotype comprising the IPMK variant nucleic acid molecule and/or the IDO2 variant nucleic acid molecule. For subjects that are IPMK reference and IDO2 reference, the methods comprise administering or continuing to administer the uveitis therapeutic agent in a standard dosage amount. For subjects that are heterozygous or homozygous for the IPMK variant nucleic acid molecule, the methods comprise administering or continuing to administer the uveitis therapeutic agent in an amount that is the same as or less than a standard dosage amount, and/or administering an IPMK therapeutic agent to the subject. For subjects that are heterozygous or homozygous for the IDO2 variant nucleic acid molecule, the methods comprise administering or continuing to administer the uveitis therapeutic agent in an amount that is the same as or less than a standard dosage amount, and/or administering an IDO2 agonist to the subject. The presence of a genotype having the IPMK variant nucleic acid molecule and/or the IDO2 variant nucleic acid molecule indicates the subject has an increased risk of developing uveitis.

For subjects that are genotyped or determined to be heterozygous or homozygous for the IPMK variant nucleic acid molecule, such subjects can be treated with an IPMK therapeutic agent, as described herein. For subjects that are genotyped or determined to be heterozygous or homozygous for the IDO2 variant nucleic acid molecule, such subjects can be treated with an IDO2 agonist, as described herein.

In some embodiments, the uveitis is anterior uveitis. In some embodiments, the uveitis is acute anterior uveitis. In some embodiments, the uveitis is pan-uveitis. In some embodiments, the subject has iridocyclitis. In some embodiments, the subject has iritis.

In some embodiments, the subject is IPMK reference and IDO2 reference, and the subject is administered or continued to be administered the uveitis therapeutic agent in a standard dosage amount. In some embodiments, the subject is heterozygous or homozygous for the IPMK variant nucleic acid molecule, and the subject is administered or continued to be administered the uveitis therapeutic agent in an amount that is the same as or less than a standard dosage amount, and the subject is administered an IPMK therapeutic agent. In some embodiments, the subject is heterozygous or homozygous for the IDO2 variant nucleic acid molecule, and the subject is administered or continued to be administered the uveitis therapeutic agent in an amount that is the same as or less than a standard dosage amount, and the subject is administered an IDO2 agonist. Any of the IPMK therapeutic agents and/or the IDO2 agonists described herein can be administered.

In some embodiments, the IPMK variant nucleic acid molecule comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A. In some embodiments, the IDO2 variant nucleic acid molecule comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C.

In some embodiments, the dose of the uveitis therapeutic agents can be reduced by about 10%, by about 20%, by about 30%, by about 40%, by about 50%, by about 60%, by about 70%, by about 80%, or by about 90% for subjects that are heterozygous or homozygous for an IPMK variant nucleic acid molecule or an IDO2 variant nucleic acid molecule (i.e., less than the standard dosage amount) compared to subjects that are IPMK reference and IDO2 reference (who may receive a standard dosage amount). In some embodiments, the dose of the uveitis therapeutic agents can be reduced by about 10%, by about 20%, by about 30%, by about 40%, or by about 50%. In addition, the dose of uveitis therapeutic agents in subjects that are heterozygous or homozygous for an IPMK variant nucleic acid molecule or an IDO2 variant nucleic acid molecule can be administered less frequently compared to subjects that are IPMK reference and IDO2 reference.

Administration of the uveitis therapeutic agents and/or IPMK therapeutic agents and/or IDO2 agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the uveitis therapeutic agents and/or IPMK therapeutic agents and/or IDO2 agonists can be administered sequentially or at the same time. In addition, the uveitis therapeutic agents and/or IPMK therapeutic agents and/or IDO2 agonists can be administered in separate compositions or can be administered together in the same composition.

Administration of the uveitis therapeutic agents and/or IPMK therapeutic agents and/or IDO2 agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in uveitis, a decrease/reduction in the severity of uveitis (such as, for example, a reduction or inhibition of development of anterior uveitis), a decrease/reduction in symptoms and uveitis-related effects, delaying the onset of symptoms of uveitis-related effects, reducing the severity of symptoms of uveitis-related effects, reducing the severity of an acute episode, reducing the number of symptoms of uveitis-related effects, reducing the latency of symptoms of uveitis-related effects, an amelioration of symptoms of uveitis-related effects, reducing secondary symptoms, reducing secondary infections, preventing relapse to uveitis, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of any therapeutic agent or composition. A prophylactic effect may comprise a complete or partial avoidance/inhibition or a delay of uveitis development/progression (such as, for example, a complete or partial avoidance/inhibition or a delay), and an increased survival time of the affected host animal, following administration of a therapeutic protocol. Treatment of uveitis encompasses the treatment of subjects already diagnosed as having any form of uveitis at any clinical stage or manifestation, the delay of the onset or evolution or aggravation or deterioration of the symptoms or signs of uveitis, and/or preventing and/or reducing the severity of uveitis.

The present disclosure also provides methods of identifying a B27-negative subject having an increased risk of developing uveitis, such as anterior uveitis. In some embodiments, the methods comprise determining or having determined the presence or absence of an IPMK variant nucleic acid molecule or an IDO2 variant nucleic acid molecule (such as a genomic nucleic acid molecule, mRNA molecule, and/or cDNA molecule) in a biological sample obtained from the subject. When the subject is IPMK reference and IDO2 reference, then the subject does not have an increased risk of developing uveitis (compared to a subject that is heterozygous or homozygous for an IPMK variant nucleic acid molecule or an IDO2 variant nucleic acid molecule). When the subject is heterozygous or homozygous for an IPMK variant nucleic acid molecule or an IDO2 variant nucleic acid molecule, then the subject has an increased risk of developing uveitis.

In some embodiments, the uveitis is anterior uveitis. In some embodiments, the uveitis is acute anterior uveitis. In some embodiments, the uveitis is pan-uveitis. In some embodiments, the subject has iridocyclitis. In some embodiments, the subject has iritis.

In some embodiments, the IPMK variant nucleic acid molecule comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A. In some embodiments, the IDO2 variant nucleic acid molecule comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C.

Detecting the presence or absence of an IPMK variant nucleic acid molecule and/or an IDO2 variant nucleic acid molecule in a biological sample obtained from a subject and/or determining whether a subject has an IPMK variant nucleic acid molecule and/or an IDO2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the IPMK variant nucleic acid molecule and/or the IDO2 variant nucleic acid molecule can be present within a cell obtained from the subject. In some embodiments, the detecting step is carried out in vitro. In some embodiments, the detecting step comprises sequencing the entire nucleic acid molecule.

In some embodiments, when the subject is IPMK reference and IDO2 reference, the subject is administered or continued to be administered the uveitis therapeutic agent in a standard dosage amount (as described herein). In some embodiments, when the subject is heterozygous or homozygous for the IPMK variant nucleic acid molecule, and the subject is administered or continued to be administered the uveitis therapeutic agent in an amount that is the same as or less than a standard dosage amount, and/or the subject is administered an IPMK therapeutic agent (as described herein). In some embodiments, when the subject is heterozygous or homozygous for the IDO2 variant nucleic acid molecule, and the subject is administered or continued to be administered the uveitis therapeutic agent in an amount that is the same as or less than a standard dosage amount, and/or the subject is administered an IDO2 agonist (as described herein).

The present disclosure also provides methods of determining a subject's aggregate burden, or risk score, of having two or more IPMK variant nucleic acid molecules and/or two or more IPMK variant polypeptides associated with an increased risk of developing uveitis, and/or of having two or more IDO2 variant nucleic acid molecules and/or two or more IDO2 variant polypeptides associated with an increased risk of developing uveitis. The aggregate burden is the sum of two or more genetic variants that can be carried out in an association analysis with uveitis. In some embodiments, the subject is homozygous for one or more IPMK variant nucleic acid molecules associated with a decreased risk of developing uveitis and/or one or more IDO2 variant nucleic acid molecules associated with a decreased risk of developing uveitis. In some embodiments, the subject is heterozygous for one or more IPMK variant nucleic acid molecules associated with a decreased risk of developing uveitis and/or one or more IDO2 variant nucleic acid molecules associated with a decreased risk of developing uveitis. When the subject has a lower aggregate burden, the subject has an decreased risk of developing uveitis, and the subject is administered or continued to be administered the uveitis therapeutic agent in an amount that is the same as or less than the standard dosage amount. When the subject has a higher aggregate burden, the subject has an increased risk of developing uveitis and the subject is administered or continued to be administered the uveitis therapeutic agent in a standard dosage amount or less than a standard dosage amount, and/or an IPMK therapeutic agent, and/or an IDO2 agonist. The higher the aggregate burden, the higher the risk of developing uveitis.

In some embodiments, a subject's aggregate burden of having any two or more IPMK variant nucleic acid molecules and/or any two or more IDO2 variant nucleic acid molecules associated with an increased risk of developing uveitis represents a weighted sum of a plurality of any of the IPMK and/or IDO2 variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the IPMK and/or IDO2 gene, where the genetic burden is the number of alleles multiplied by the association estimate with uveitis or related outcome for each allele (e.g., a weighted polygenic burden score). In some embodiments, when the subject has an aggregate burden higher than a desired threshold score, the subject has an increased risk of developing uveitis. In some embodiments, when the subject has an aggregate burden lower than a desired threshold score, the subject has a decreased risk of developing uveitis.

In some embodiments, the aggregate burden may be divided into quintiles, e.g., top quintile, second quintile, intermediate quintile, fourth quintile, and bottom quintile, wherein the top quintile of aggregate burden corresponds to the highest risk group and the bottom quintile of aggregate burden corresponds to the lowest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with uveitis in the top 10%, top 20%, top 30%, top 40%, or top 50% of p-value range for the association. In some embodiments, each of the identified genetic variants comprise the genetic variants having association with uveitis with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with uveitis with p-value of less than 5×10⁻⁸. In some embodiments, the identified genetic variants comprise genetic variants having association with uveitis in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the top decile, quintile, or tertile in a reference population. The threshold of the aggregate burden can be determined on the basis of the nature of the intended practical application and the risk difference that would be considered meaningful for that practical application.

In embodiments where the aggregate burden is determined for IPMK genetic variants associated with uveitis and/or IDO2 genetic variants associated with uveitis, then the aggregate burden represents a subject's risk score for developing uveitis. In some embodiments, the aggregate burden or risk score includes the IPMK variant genomic nucleic acid molecule that comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A, or is an mRNA molecule produced therefrom, or is a cDNA molecule produced from the mRNA molecule. In some embodiments, the aggregate burden or risk score includes the IDO2 variant genomic nucleic acid molecule that comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C, or is an mRNA molecule produced therefrom, or is a cDNA molecule produced from the mRNA molecule. In some embodiments, a subject's aggregate burden can be determined for IPMK genetic variants associated with uveitis in combination with IDO2 genetic variants associated with uveitis (or with any additional genetic variants for other genes also associated with uveitis) to produce a polygenic risk score (PRS) for developing uveitis.

In some embodiments, the uveitis is anterior uveitis. In some embodiments, the uveitis is acute anterior uveitis. In some embodiments, the uveitis is pan-uveitis. In some embodiments, the subject has iridocyclitis. In some embodiments, the subject has iritis.

The present disclosure also provides methods of detecting the presence or absence of an IPMK variant nucleic acid molecule and/or an IDO2 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any IPMK variant nucleic acid molecule and/or any IDO2 variant nucleic acid molecule, preliminary processing designed to isolate or enrich the biological sample for the genomic DNA can be employed. A variety of techniques may be used for this purpose. When detecting the level of any IPMK variant nucleic acid molecule and/or any IDO2 variant nucleic acid molecule variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an IPMK variant nucleic acid molecule and/or an IDO2 variant nucleic acid molecule in a subject comprises performing a sequence analysis on a biological sample obtained from the subject to determine whether an IPMK variant genomic nucleic acid molecule and/or an IDO2 variant genomic nucleic acid molecule genomic nucleic acid molecule in the biological sample, and/or an IPMK variant mRNA molecule and/or an IDO2 variant mRNA molecule in the biological sample, and/or an IPMK variant cDNA molecule produced from an mRNA molecule in the biological sample and/or an IDO2 variant cDNA molecule produced from an mRNA molecule in the biological sample, is present in the sample. In some embodiments, the methods detect an IPMK variant genomic nucleic acid molecule that comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A, or is an mRNA molecule produced therefrom, or is a cDNA molecule produced from the mRNA molecule. In some embodiments, the methods detect an IDO2 variant genomic nucleic acid molecule that comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C, or is an mRNA molecule produced therefrom, or is a cDNA molecule produced from the mRNA molecule.

In some embodiments, the methods of detecting the presence or absence of an IPMK variant nucleic acid molecule and/or an IDO2 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an IPMK variant genomic nucleic acid molecule and/or an IDO2 variant genomic nucleic acid molecule, or mRNA molecule, and if mRNA, optionally reverse transcribing the mRNA into cDNA. Such assays can comprise, for example determining the identity of these positions of the particular IPMK variant nucleic acid molecule and/or IDO2 variant nucleic acid molecule. In some embodiments, the method is an in vitro method.

In some embodiments, the determining step, detecting step, or sequence analysis comprises sequencing at least a portion of the nucleotide sequence of the IPMK variant genomic nucleic acid molecule and/or the IDO2 variant genomic nucleic acid molecule, the IPMK variant mRNA molecule and/or the IDO2 variant mRNA molecule, or the IPMK variant cDNA molecule and/or the IDO2 variant cDNA molecule in the biological sample that comprises a genetic variation compared to the corresponding IPMK and/or IDO2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an IPMK variant genomic nucleic acid molecule and/or an IDO2 variant genomic nucleic acid molecule is analyzed. In some embodiments, only an IPMK variant mRNA molecule and/or an IDO2 variant mRNA molecule is analyzed. In some embodiments, only an IPMK variant cDNA molecule and/or an IDO2 variant cDNA molecule from the mRNA molecule is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject. In some embodiments, the assay comprises contacting the biological sample with a primer or probe, such as an alteration-specific primer or alteration-specific probe, that specifically hybridizes to an IPMK variant genomic nucleic acid molecule or an IDO2 variant genomic nucleic acid molecule, variant mRNA sequence, or variant cDNA sequence and not the corresponding an IPMK or IDO2 reference sequence under stringent conditions and determining whether hybridization has occurred.

In some embodiments, the determining step, detecting step, or sequence analysis comprises: a) amplifying at least a portion of the IPMK variant nucleic acid molecule and/or the IDO2 variant nucleic acid molecule that encodes the IPMK and/or the IDO2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an IPMK variant genomic nucleic acid molecule and/or an IDO2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to IPMK variant nucleic acid molecules and/or IDO2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to IPMK variant nucleic acid molecules or an IDO2 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the IPMK variant nucleic acid molecules and/or any of the IDO2 variant nucleic acid molecules disclosed herein. The primers described herein can be used to amplify any IPMK variant nucleic acid molecule and/or any IDO2 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an IPMK reference genomic nucleic acid molecule and/or an IDO2 reference genomic nucleic acid molecule, an IPMK reference mRNA molecule and/or an IDO2 reference mRNA molecule, and/or an IPMK reference cDNA molecule and/or an IDO2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides uveitis therapeutic agents for use in the treatment or prevention of uveitis in a subject that is B27-negative, wherein the subject is identified as having an IPMK variant nucleic acid molecule and/or an IDO2 variant nucleic acid molecule. Any of the uveitis therapeutic agents described herein can be used herein. Any of the IPMK and/or IDO2 variant nucleic acid molecules disclosed herein can be used herein. In some embodiments, the IPMK variant nucleic acid molecule is an IPMK genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule. In some embodiments, the IDO2 variant nucleic acid molecule is an IDO2 genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule.

The present disclosure also provides uveitis therapeutic agents for use in the preparation of a medicament for treating or preventing uveitis in a subject that is B27-negative, wherein the subject is identified as having an IPMK variant nucleic acid molecule and/or an IDO2 variant nucleic acid molecule. Any of the uveitis therapeutic agents described herein can be used herein. Any of the IPMK and/or IDO2 variant nucleic acid molecules disclosed herein can be used herein. In some embodiments, the IPMK variant nucleic acid molecule is an IPMK genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule. In some embodiments, the IDO2 variant nucleic acid molecule is an IDO2 genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule.

The present disclosure also provides IPMK therapeutic agents for use in the treatment or prevention of uveitis in a B27-negative subject that comprises an IPMK variant nucleic acid molecule. Any of the IPMK therapeutic agents described herein can be used herein. Any of the IPMK variant nucleic acid molecules disclosed herein can be used herein. In some embodiments, the IPMK variant nucleic acid molecule is an IPMK variant genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule.

The present disclosure also provides IPMK therapeutic agents for use in the preparation of a medicament for treating or preventing uveitis in a B27-negative subject that comprises an IPMK variant nucleic acid molecule. Any of the IPMK therapeutic agents described herein can be used herein. Any of the IPMK variant nucleic acid molecules disclosed herein can be used herein. In some embodiments, the IPMK variant nucleic acid molecule is an IPMK variant genomic nucleic acid molecule that comprises the genetic variation 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, or 10:58227082:T:A, or is an mRNA molecule produced therefrom, or is a cDNA molecule produced from the mRNA molecule.

The present disclosure also provides IDO2 agonists for use in the treatment or prevention of uveitis in a B27-negative subject that comprises an IDO2 variant nucleic acid molecule. Any of the IDO2 agonists described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule.

The present disclosure also provides IDO2 agonists for use in the preparation of a medicament for treating or preventing uveitis in a B27-negative subject that comprises an IDO2 variant nucleic acid molecule. Any of the IDO2 agonists described herein can be used herein. Any of the IDO2 variant nucleic acid molecules disclosed herein can be used herein. In some embodiments, the IDO2 variant nucleic acid molecule is an IDO2 variant genomic nucleic acid molecule that comprises the genetic variation 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, or 8:40015454:CAGCCAAGGCAA:C, or is an mRNA molecule produced therefrom, or is a cDNA molecule produced from the mRNA molecule.

In some embodiments, the IPMK therapeutic agent, the IDO2 agonist, and the uveitis therapeutic agent are disposed within a pharmaceutical composition. In some embodiments, the IPMK therapeutic agent and/or the IDO2 agonist is disposed within a first pharmaceutical composition and the uveitis therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methodology

### Sequencing and Genotyping

Genotyping and exome sequencing were performed as previously described (Verweij et al., N. Engl. J. Med., 2022, 387, 332-344). In short, for analyses of common variants, array genotyping data was used and imputation was performed with the use of the TOPMed panel (Taliun et al., Nature, 2021, 590, 290-299) in all cohorts analyzed. Exome sequencing for rare variant analysis was performed with the use of Illumina HiSeq 2500-v4 or Illumina NovaSeq instruments, with 75-bp paired-end reads (Taliun et al., Nature, 2021, 590, 290-299). Deleterious variants were considered if annotated as: frameshift, stop-gain, stop-loss, splice acceptor, splice donor, in-frame insertion or deletion (indel), missense, and other annotations. Frameshift, stop-gain, stop-loss, splice-acceptor, and splice-donor alleles were categorized as predicted loss-of-function variants. The alternative-allele frequency and functional annotation of each variant were used to generate seven genotypes based on the combined variant burden: predicted loss-of-function variants with an alternative-allele frequency thresholds of 1% and 0.1%, predicted loss-of-function variants plus missense variants that were predicted to be deleterious and had an alternative-allele frequency thresholds of 1% and 0.1%.

### Samples

Genome-wide association analyses were performed in the U.K. Biobank population-based cohort, and in the Geisinger Health System MyCode cohort. Other datasets: 29,237 from the Malmo Diet and Cancer Study, 41,400 participants from the University of Pennsylvania Penn Medicine BioBank, 29,845 participants from the Mount Sinai BioMe BioBank, 49,004 from the Colorado cohort, 40,197 from the UCLA cohort, and 115,418 participants MAYO-clinic cohort. 829,810 participants of European ancestry, 42,688 of African ancestry, 13,866 of South Asian ancestry, 9,303 of East Asian ancestry, 18,862 with ancestry from the Americas, and 5,652 of other ancestries were included, for whom genotyping, exome-sequencing data and phenotype data were available. Cases were selected based on having the “ICD10: H20 Iridocyclitis” diagnosis code, while controls were selected if they had negative H20 code.

### Exome Sequencing and Whole-Genome Genotyping

For analyses of common variants, array genotyping data was used and imputation was performed with the use of the TOPMed reference panel (Taliun et al., bioRxiv, 563866, doi:10.1101/563866, 2019; and Das et al., Nat. Genet., 2016, 48, 1284-1287). High-coverage exome sequencing was performed with the use of Illumina HiSeq 2500-v4 or Illumina NovaSeq instruments, with 75-bp paired-end reads. The GRCh38 human genome reference sequence and Ensembl, version 85, gene definitions were used for variant identification and annotation. Variants were classified from most to least deleterious in the following order: frameshift, stop-gain, stop-loss, splice acceptor, splice donor, in-frame insertion or deletion (indel), missense, and other annotations. Frameshift, stop-gain, stop-loss, splice-acceptor, and splice-donor alleles were categorized as predicted loss-of-function variants. Missense variants were classified using computer modeling to predict functional effects with five algorithms: SIFT, Polyphen-2 HDIV, Polyphen-2 HVAR, LRT, and MutationTaster. To account for the fact that different genes have different types and frequencies of potentially causative variants, the alternative-allele frequency and functional annotation of each variant was used to generate seven genotypes based on the combined variant burden: predicted loss-of-function variants; predicted loss-of-function variants plus missense variants that were predicted to be deleterious by five of five algorithms; predicted loss-of-function variants plus missense variants that were predicted to be deleterious by at least one of five algorithms; predicted loss-of-function variants plus any missense variants; missense variants that were predicted to be deleterious by five of five algorithms; missense variants that were predicted to be deleterious by at least one of five algorithms; and finally, any missense variants at all (these categories are similar to those used previously) (Verweij et al., N. Engl. J. Med., 2022, 387, 332-344).

### Statistical Analysis

Associations between genotypes and phenotypes was estimated by fitting linear regression models (for quantitative traits) or Firth bias-corrected logistic regression models (for binary traits) using REGENIE software, version 2+(Mbatchou et al., Nat. Genet., 2021, 53, 1097-1103). Analyses were stratified according to cohort and ancestry and were adjusted for age, age squared, sex, age-by-sex, and age squared-by-sex interaction terms; experimental batch-related covariates; the first 10 common variant-derived genetic principal components; the first 20 rare variant-derived principal components; and a polygenic score generated by REGENIE, which robustly adjusts for relatedness and population structure (Mbatchou et al., Nat. Genet., 2021, 53, 1097-1103). A meta-analysis of association results across cohorts and ancestries was performed with a fixed-effect inverse-variance-weighted approach.

For gene burden analyses, each of the variant-burden categories mentioned above were tested at four thresholds of alternate-allele frequencies: alternative-allele frequencies of less than 1%; alternative-allele frequencies of less than 0.5%; alternative-allele frequencies of less than 0.1%; and alternative-allele frequencies of less than 0.01%. These seven categories and four thresholds produce 28 pseudo-genotypes for each gene, but they are not fully independent of one another, given the overlapping annotations and frequency thresholds. Thus, an appropriate adjusted Bonferroni significance level was calculated for these variant-burden tests, using a method recommended by a review of multiple-testing correction methods in non-independent genetic tests (Li et al., Heredity (Edinb), 2005, 95, 221-227; and Wen et al., J. Hum. Genet., 2011, 56, 428-435). Calculating the effective number of independent tests based on the correlation matrix of these variant-burden tests in the meta-analysis resulted in a value of 9.002158 tests per gene, which, when multiplied by the number of genes tested (19,446) and used as a correction factor for an alpha level of 0.05, resulted in an exome-wide level of significance at a P value of 2.86e-07.

### Example 2: Risk Alleles Associating with B27-Negative AU

### Common Genetic Signals Contributing to AU Risk

In order to assemble the largest AU case cohort to date, eight large EHR-based populations, reaching 3,850 AU cases and 916,462 controls (see, Table 1) were sequenced. Testing the association of common variants, two genome wide significant signals were discovered: 1) a risk signal for rs543685299 at the HLA-B locus (OR[95% CI]=3.37 [3.12-3.65], p=7.82E-197), and 2) a signal for rs3198304 at the ERAP1 locus (OR[95% CI]=0.84 [0.79-0.89], p=5.02e-9) that presents protection from AU (data not shown). The top ERAP1 SNP showed a replicated direction of protection in 6/8 cohorts (data not shown) and was found significantly protective in the two larger cohorts of UKB and GHS.

TABLE 1
Overview of cohorts included in the meta-analysis

| | EUR | | ALL | |
|---|---|---|---|---|
| | Cases | Controls | Cases | Controls |
| Cohort | (B-27; %) | (B-27; %) | (B-27; %) | (B-27; %) |
| UKB 450k | 1,260 | 429,728 | 1,388 | 452,976 |
| | (420; 33.3%) | (35,066; 8.2%) | (424; 30.5%) | (35,464; 7.8%) |
| GHS175K | 1,007 | 150,547 | 1,066 | 159,644 |
| | (184; 18.3%) | (12,205; 8.1%) | (188; 17.6%) | (12,484; 7.8%) |
| UPenn- | 75 | 28,426 | 233 | 41,304 |
| PMBB | (13; 17.33%) | (2209; 7.7%) | (19; 8.1%) | (2434; 5.9%) |
| Sinai | 51 | 10,681 | 169 | 29,676 |
| | (13; 25.5%) | (603; 5.6%) | (20; 11.8%) | (1,086; 3.7%) |
| MALMO | 114 | 28,834 | 116 | 29,121 |
| | (30; 26.3%) | (2,807; 9.7%) | (30; 25.9%) | (2,818; 9.7%) |
| MAYO- | 331 | 110,930 | 355 | 115,063 |
| Clinic | (72; 21.8%) | (9,711; 8.8%) | (74; 20.8%) | (9,902; 8.6%) |
| UCLA | 161 | 26,276 | 305 | 39,892 |
| | (36; 22.4%) | (1,880; 7.2%) | (54; 17.8%) | (2,306; 5.8%) |
| Colorado | 181 | 40,980 | 218 | 48,786 |
| | (40; 22.1%) | (3356; 8.2%) | (47; 21.6%) | (3698; 7.6%) |
| Total | 3,180 | 826,402 | 3,850 | 916,462 |
| | (796; 25.0%) | (66,109; 8.0%) | (844; 21.9%) | (68,359; 7.5%) |

The analysis was repeated using an EUR-only cohort of 3,180 EUR cases and 826,348 controls. In this less powered analysis, 17% of total cases and 10% of controls were removed, yet similar signals were found for both HLA-B (OR=3.4, p=9.7e-186) and ERAP1 (OR=0.83, p=3.2e-09).

### Gene Burden Analyses of Rare Variants Identify Risk Genes Contributing to AU Risk

The gene burden analysis of all cohorts, combining all collapsing models showed a controlled low inflation of λ=0.94, allowing for the confident identification of genes that pass the strict genome-wide significant threshold of p=2.86e-07.

Two additional genes passed the genome-wide study-wide significance threshold of p=2.86e-07 multiple testing correction that incorporates all genes, variant-burden categories, and their correlation matrix (Li et al., Heredity (Edinb), 2005, 95, 221-227). The first, IPMK, is significant when considering a model that includes pLoF and missense variants that are strongly deleterious (predicted by 5/5 prediction models), with AF<0.1%, reaching a high OR (95% CI)=9.42 (4.44-19.89) with a strong p=4.4e-09 (see, Table 2). When considering a stricter singleton mask, requiring each variant to appear one time in each cohort, IPMK was still genome-wide significant, increasing the OR to 18.9 (6.98-51.22) with a weaker p=7.46e-09). The top association, consisting of 11 AU case carriers and 411 controls, was obtained from the aggregation of all cohorts, but only four of which contained case carriers. The variants that constitute the mask included ten distinct pLoF and missense variants (6 missense, 2 stop-gain, 2 frame-shifts), none of which were significant independently (data not shown).

The second genome-wide and study-wide significant gene was IDO2 that showed a strong risk signal when considering only rare (AF<0.1%) pLoF variants only, exhibiting an OR (95% CI)=3.61 (2.23-5.7) and p=6.16e-08. The direction of risk was maintained but weakened when considering more common pLoF variants and/or the addition of missense variants. The top association, consisting of 28 AU cases (27 Het and 1 Hom) and 1438 controls (1433/5), was obtained from the aggregation of all cohorts, seven of which contained case carriers. The variants that constitute the mask included seven distinct pLoF variants, four stop-gain, two frame-shifts and one splice-donor, none of which were significant by themselves (data not shown).

TABLE 2
Top gene burden results for AU

| | Burden | | | Cases | Controls | Firth | FET OR; |
|---|---|---|---|---|---|---|---|
| Gene | mask | AF | Effect | (ref \| het \| alt) | (ref \| het \| alt) | Pval | Pval |
| IPMK | pLoF and | <0.1% | 9.40 | 3839 \| 11 \| 0 | 916138 \| 411 \| 0 | 4.42E−09 | OR = 6.39; |
| | damaging | | | | | | p = 2.35e−06 |
| | missense | | | | | | |
| | (5/5) | | | | | | |
| IDO2 | pLoF only | <0.1% | 3.61 | 3822 \| 27 \| 1 | 915111 \| 1433 \| 5 | 6.16E−08 | OR = 4.66; |
| | | | | | | | p = 8.18e−11 |

### Controlling for HLA-B27 Carrier Status

To better understand the genetic signals underlying HLA-B27 in the AU cohort, an HLA-B27 tag SNP rs4349859 as a covariate was used to control for HLA-B27 in the analyses. When controlling for this SNP, the HLA-B27 signal diminished leaving a residual borderline signal on HLA-DPB1 (OR=1.15, p=6e-08). A novel genome-wide significant common signal near HLA-DPB1 (rs3117230, OR=1.26, p=2.7e-08) was identified. The protective signal on ERAP1 remained genome-wide significant (OR=0.84, p=1.7e-8). Following this analysis, it was uncertain whether the ERAP1 and remaining HLA signal were specifically associated with HLA-B27 carrier status of the AU cases. It was therefore decided to further stratify the datasets and individually analyze the carriers and non-carriers of HLA-B27 allele.

In particular, a stratified analyses was designed by which the cohorts were divided by the carrier status of HLA-B27 using the HLA-B27 tag SNP rs4349859. The HLA-B27 stratification resulted in two cohorts: 1) a B27-positive cohort with samples carrying either one or two copies of the tag SNP, and 2) a B27-negative cohort with samples carrying zero copies of the tag SNP.

### B27-Negative AU

### HLA-DPB1 is a Significant Risk for B27-Negative AU

An analysis was performed including only cases and control that do not carry the B27 tag SNP, which was considered as a B27-negative AU. This cohort consisted of 2,806 B27-negative AU cases and 794,576 B27-negative controls. The analysis presented a novel genome-wide significant signal at rs6914651, an HLA-Class-II gene region near HLA-DPB1, which was not previously associated with AU [OR=1.18 (1.11-1.25), p=1.6e-08]. Since having an allele frequency of 0.277, rs6914651 is a common signal that might reflect a coding region within HLA-DPB1 that is associated with AU risk, which in turn might point to a specific HLA-DPB1 allele that increases risk of HLA-B27 negative AU. To answer this question, two additional analyses were carried out: 1) imputing the HLA-DPB1 alleles and testing for association of each allele with case-control status, and 2) fine-mapping of the region near HLA-DPB1 to uncover the genetic signals that underlie this significant association. The results of testing the associations of class-II HLA alleles have shown HLA-DPB1*04:01 as a protective allele (p=2.3e-05, OR=0.89) and HLA-DPB1*03:01 as risk (p1.4e-03, OR=1.19). However, when adjusting for the top SNP (rs6914651) in the regression model, neither HLA-DPB1*03:01 or HLA-DPB1*04:01 are nominally significant, suggesting that it is not a specific HLA-DPB1 allele that affecting AU risk (data not shown). Furthermore, the results of fine-mapping the DPB1 region also suggested that the signal originates not from HLA-DPB1 itself, but from the region downstream to HLA-DPB1, where a long stretch of non-coding variants share similar posterior inclusion probabilities (data not shown). Last, rs6914651 acts as an eQTL for HLA-DPB1 significantly decreasing its expression, suggesting that the effect might lie in the top signal affecting AU risk by decreasing HLA-DPB1 expression (Consortium, Science, 2020, 369, 1318-1330). This, in turn, might translate into HLA-DPB1*04:01 exhibiting protection and DPB1*03:01 exhibiting risk, when SNPs that are shared but reversed in both alleles are in close proximity and, thus, in LD with the top SNP.

### Gene Burden Analyses of B27-Negative AU

Whether the gene burden results using the B27-negative AU cohort described above replicate the analyses using all samples was analyzed. This question is highly relevant to deciphering the mechanism underlying both sub-types of AU. It was found that both IPMK and IDO2 replicate a similar direction of risk in the B27-negative cohort: the same IPMK model that includes pLoF and missense variants that are strongly deleterious (predicted by 5/5 prediction models), with AF<0.1%, exhibits a similar significance with OR (95% CI)=10.32 (4.5-22.2) and p=2.32e-09. This is due to the previous eleven case carriers all belonging to the B27-negative group, while control samples are decreased to 381 (see, Table 3, Table 4, Table 5). For IDO2, the rare pLoF mask (AF<0.1%) exhibits a similar effect with OR=3.27 (2.04-5.26), but a weaker p=9.8e-07 owing to the decreased power. Both of those genes do not show significant associations in the B27-positive cohort, suggesting these mechanisms of risk pertain to B27-negative AU.

TABLE 3
IPMK and IDO2 genes associate with B27-negative AU (all ancestries)

| | Burden | | | Effect | | Cases | Controls |
|---|---|---|---|---|---|---|---|
| Gene | mask | AF | Cohort | (LCI-UCI) | Pval | (ref \| het \| alt) | (ref \| het alt) |
| IPMK | pLoF and | <0.1% | Full | 9.4 | 4.42E−09 | 3839 \| 11 \| 0 | 916138 \| 411 \| 0 |
| | damaging | | cohort | (4.5-19.9) | | | |
| | missense | | B27-neg | 10.32 | 2.32E−09 | 2973 \| 11 \| 0 | 844331 \| 381 \| 0 |
| | (5/5) | | cohort | (4.8-22.2) | | | |
| | | | B27- | 0.68 | 1.99E−01 | 848 \| 8 \| 0 | 69115 \| 989 \| 5 |
| | | | positive | (0.4-1.2) | | | |
| | | | cohort | | | | |
| IDO2 | pLoF only | <0.1% | Full | 3.61 | 6.16E−08 | 3822 \| 27 \| 1 | 915111 \| 1433 \| 5 |
| | | | cohort | (2.3-5.8) | | | |
| | | | B27-neg | 3.27 | 9.67E−07 | 2958 \| 25 \| 1 | 843325 \| 1382 \| 5 |
| | | | cohort | (2.0-5.3) | | | |
| | | | B27- | 9.56 | 2.31E−02 | 824 \| 2 \| 0 | 67243 \| 48 \| 0 |
| | | | positive | (1.4-67.0) | | | |
| | | | cohort | | | | |

TABLE 4
IPMK and IDO2 genes associate with B27-negative AU (EUR only)

| | Burden | | | Effect | | Cases | Controls |
|---|---|---|---|---|---|---|---|
| Gene | mask | AF | Cohort | (LCI-UCI) | Pval | (ref \| het \| alt) | (ref \| het \| alt) |
| IPMK | pLoF and | <0.1% | Full | 8.04 | 7.71E−05 | 3174 \| 6 \| 0 | 826121 \| 336 \| 0 |
| | damaging | | cohort | (2.9-22.6) | | | |
| | missense | | B27-neg | 11.32 | 1.25E−05 | 2357 \| 6 \| 0 | 756860 \| 308 \| 0 |
| | (5/5) | | cohort | (3.8-33.6) | | | |
| | | | B27- | 0.34 | 5.47E−01 | 787 \| 0 \| 0 | 63821 \| 30 \| 0 |
| | | | positive | (0.01-11.4) | | | |
| | | | cohort | | | | |
| IDO2 | pLoF only | <0.1% | Full | 2.96 | 2.30E−02 | 3174 \| 6 \| 0 | 825771 \| 686 \| 0 |
| | | | cohort | (1.2-7.6) | | | |
| | | | B27-neg | 2.32 | 1.10E−01 | 2359 \| 4 \| 0 | 756525 \| 643 \| 0 |
| | | | cohort | (0.8-6.5) | | | |
| | | | B27- | 7.21 | 6.64E−02 | 776 \| 2 \| 0 | 64911 \| 43 \| 0 |
| | | | positive | (0.9-59.5) | | | |
| | | | cohort | | | | |

TABLE 5
IPMK and IDO2 genes associate with B27-negative AU (AFR only)

| | Burden | | | Effect | | Cases | Controls |
|---|---|---|---|---|---|---|---|
| Gene | mask | AF | Cohort | (LCI-UCI) | Pval | (ref \| het \| alt) | (ref \| het \| alt) |
| IPMK | pLoF and | <0.1% | Full | 21.14 | 4.64E−04 | 417 \| 3 \| 0 | 42243 \| 27 \| 0 |
| | damaging | | cohort | (3.8-116.0) | | | |
| | missense | | B27-neg | 25.49 | 2.24E−04 | 400 \| 3 \| 0 | 41409 \| 27 \| 0 |
| | (5/5) | | cohort | (4.6-142.4) | | | |
| | | | B27- | NA | | | |
| | | | positive | | | | |
| | | | cohort | | | | |
| IDO2 | pLoF only | <0.1% | Full | 2.15 | 1.93E−03 | 400 \| 19 \| 1 | 41598 \| 667 \| 5 |
| | | | cohort | (1.3-3.5) | | | |
| | | | B27-neg | 2.32 | 9.12E−04 | 383 \| 19 \| 1 | 40771 \| 660 \| 5 |
| | | | cohort | (1.4-3.8) | | | |
| | | | B27- | | | | |

| positive | NA |
|---|---|
| cohort | |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating uveitis, iridocyclitis, or iritis in a subject that is HLA-B27-negative, the method comprising administering an Inositol Polyphosphate Multikinase (IPMK) therapeutic agent or an Indoleamine 2,3-dioxygenase 2 (IDO2) agonist.

2. The method of claim 1, wherein the uveitis is anterior uveitis.

3. The method of claim 1, wherein the uveitis is acute anterior uveitis.

4. The method of claim 1, wherein the uveitis is pan-uveitis.

5-6. (canceled)

7. The method of claim 1, wherein the IPMK therapeutic agent comprises IPMK protein.

8. The method of claim 1, wherein the IDO2 agonist comprises IDO2 protein.

9. The method of claim 1, further comprising administering a uveitis therapeutic agent to the subject.

10. The method of claim 1, further comprising detecting the presence or absence of an IPMK variant nucleic acid molecule and/or a IDO2 variant nucleic acid molecule in a biological sample obtained from the subject.

11. The method of claim 10, further comprising administering a uveitis therapeutic agent in a standard dosage amount to a subject wherein the IPMK variant nucleic acid molecule is absent from the biological sample.

12. The method of claim 10, further comprising administering a uveitis therapeutic agent in a dosage amount that is the same as or less than a standard dosage amount to a subject that is heterozygous or homozygous for the IPMK variant nucleic acid molecule.

13. The method of claim 10, further comprising administering a uveitis therapeutic agent in a standard dosage amount to a subject wherein the IDO2 variant nucleic acid molecule is absent from the biological sample.

14. The method of claim 10, further comprising administering a uveitis therapeutic agent in a dosage amount that is the same as or less than a standard dosage amount to a subject that is heterozygous or homozygous for the IDO2 variant nucleic acid molecule.

15. The method of claim 10, wherein the IPMK variant nucleic acid molecule comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A.

16. The method of claim 10, wherein the IDO2 variant nucleic acid molecule comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C.

17. The method of claim 10, wherein the detecting step is carried out in vitro.

18. The method of claim 10, wherein the detecting step comprises sequencing the entire nucleic acid molecule.

19. A method of treating a subject with a uveitis therapeutic agent, wherein the subject has B27-negative uveitis, the method comprising:
determining whether the subject has an Inositol Polyphosphate Multikinase (IPMK) variant nucleic acid molecule or an Indoleamine 2,3-dioxygenase 2 (IDO2) variant nucleic acid molecule by:
obtaining or having obtained a biological sample from the subject; and
performing or having performed a sequence analysis on the biological sample to determine if the subject has a genotype comprising the IPMK variant nucleic acid molecule and/or the IDO2 variant nucleic acid molecule; and
administering or continuing to administer the uveitis therapeutic agent in a standard dosage amount to a subject that is IPMK reference and IDO2 reference;
administering or continuing to administer the uveitis therapeutic agent in an amount that is the same as or less than a standard dosage amount to a subject that is heterozygous or homozygous for the IPMK variant nucleic acid molecule, and/or administering an IPMK therapeutic agent to the subject; and
administering or continuing to administer the uveitis therapeutic agent in an amount that is the same as or less than a standard dosage amount to a subject that is heterozygous or homozygous for the IDO2 variant nucleic acid molecule, and/or administering an IDO2 agonist to the subject;
wherein the presence of a genotype having the IPMK variant nucleic acid molecule and/or the IDO2 variant nucleic acid molecule indicates the subject has an increased risk of developing uveitis.

20. The method of claim 19, wherein the uveitis is anterior uveitis, acute anterior uveitis, or pan-uveitis.

21-22. (canceled)

23. The method of claim 19, wherein the subject has iridocyclitis or iritis.

24. (canceled)

25. The method of claim 19, wherein the subject is IPMK reference and IDO2 reference, and the subject is administered or continued to be administered the uveitis therapeutic agent in a standard dosage amount.

26. The method of claim 19, wherein the subject is heterozygous or homozygous for the IPMK variant nucleic acid molecule, and the subject is administered or continued to be administered the uveitis therapeutic agent in an amount that is the same as or less than a standard dosage amount, and the subject is administered an IPMK therapeutic agent.

27. The method of claim 19, wherein the subject is heterozygous or homozygous for the IDO2 variant nucleic acid molecule, and the subject is administered or continued to be administered the uveitis therapeutic agent in an amount that is the same as or less than a standard dosage amount, and the subject is administered an IDO2 agonist.

28. The method of claim 19, wherein the IPMK therapeutic agent comprises IPMK protein.

29. The method of claim 19, wherein the IDO2 agonist comprises IDO2 protein.

30. The method of claim 19, wherein the IPMK variant nucleic acid molecule comprises at least one of the genetic variations 10:58196183:G:A, 10:58196186:C:A, 10:58196330:TCTGA:T, 10:58196405:ACT:A, 10:58196488:T:A, 10:58196488:T:A, 10:58196488:T:G, 10:58196512:G:A, 10:58216170:A:G, 10:58216302:A:T, and 10:58227082:T:A.

31. The method of claim 19, wherein the IDO2 variant nucleic acid molecule comprises at least one of the genetic variations 8:39935219:G:T, 8:39987871:GA:G, 8:39989787:C:T, 8:39989787:C:T, 8:39989787:C:T, 8:39989787:C:T, 8:39989793:A:T, 8:39989793:A:T, 8:39989793:A:T, 8:39989793:A:T, 8:40013653:C:T, 8:40015381:C:T, 8:40015381:C:T, and 8:40015454:CAGCCAAGGCAA:C.

32. The method of claim 19, wherein the determining step is carried out in vitro.

33. The method of claim 19, wherein the determining step comprises sequencing the entire nucleic acid molecule.

34-53. (canceled)
